(19) United States
(12) Patent Application Publication
(10) Pub. No.: US 2026/0165581 A1
(43) Pub. Date: Jun. 18, 2026

(54) Structured Light-Based Method and Portable Device for Early Detection of Myopia and Other Refractive Errors Using Modulation Transfer Function (MTF) and Liquid Lens Adjustment

(71) Applicant: Mohsen Sharifzadeh, Cottonwood Heights, UT (US)

(72) Inventor: Mohsen Sharifzadeh, Cottonwood Heights, UT (US)

(21) Appl. No.: 19/365,013

(22) Filed: Oct. 21, 2025

Related U.S. Application Data
(60) Provisional application No. 63/734,181, filed on Dec. 15, 2024.

(51) Int. Cl. A61B 3/14; A61B 3/00; A61B 3/103; G06T 7/00; G06T 7/262

(52) U.S. Cl. CPC A61B 3/14; A61B 3/0025; A61B 3/005; A61B 3/103; G06T 7/0012; G06T 7/262; G06T 2207/30041

(57) ABSTRACT

The invention provides a structured light-based method and portable device for measuring ocular axial length and refractive error using Modulation Transfer Function (MTF) analysis and liquid lens adjustment. A polarized structured light pattern is projected onto the retina, and the reflected image is captured through a cross-polarization filter to isolate retinal reflections. A liquid lens dynamically adjusts focus while the MTF is computed for each focal setting to quantify image sharpness. The focal position corresponding to maximum MTF indicates the optical conjugate of the retinal plane, enabling estimation of refractive error and axial length using a calibrated optical model. Optional deformation metrics, artificial intelligence modules, and telemedicine connectivity enhance robustness and accessibility. The invention provides a compact, non-invasive, and cost-effective system for early detection and monitoring of myopia and other refractive conditions.

### CROSS-REFERENCE TO RELATED APPLICATIONS

This application claims the benefit of priority under 35 U.S.C. § 119 (e) to U.S. Provisional Patent Application No. 63/734,181, filed Dec. 15, 2024, entitled “Structured Light-Based Method and Device for Measuring Ocular Axial Length and Refractive Error Using Modulation Transfer Function (MTF) and Liquid Lens Adjustment.”

The entire disclosure of the above-referenced provisional application is hereby incorporated by reference in its entirety.

### FIELD OF THE INVENTION

The present invention relates generally to ophthalmic diagnostic systems and methods, and more particularly to a structured light-based optical method and portable device for measuring ocular axial length and refractive error.

Specifically, the invention combines structured light projection, Modulation Transfer Function (MTF) analysis, and liquid lens adjustment to enable non-invasive, real-time estimation of axial length and refractive power of the human eye.

The invention is applicable to early detection and monitoring of myopia and other refractive conditions, particularly in pediatric and resource-limited settings, where conventional optical coherence tomography (OCT) or ultrasound-based devices are impractical due to cost, size, or complexity.

### BACKGROUND OF THE INVENTION

Accurate measurement of ocular axial length and refractive error is essential for diagnosing and managing myopia, hyperopia, and other refractive disorders.

Existing diagnostic instruments, such as ultrasound biometry, optical low-coherence interferometry, and optical coherence tomography (OCT), provide precise axial length measurements but suffer from several limitations:
1. High Cost and Complexity: Conventional devices rely on interferometric or ultrasound systems that require precise alignment, trained operators, and expensive components, limiting accessibility in primary care or community settings.
2. Limited Portability: These systems are typically large, immobile instruments, unsuitable for school-based or point-of-care screening programs.
3. Invasiveness and Patient Discomfort: Contact ultrasound requires corneal coupling and can cause patient discomfort or operator variability.
4. Restricted Use in Pediatric Populations: Children may struggle with fixation and positioning required for conventional imaging systems, compromising measurement accuracy.

Given the global rise of pediatric myopia, expected to affect nearly half the world's population by 2050, there is an urgent need for low-cost, non-invasive, and portable solutions for early detection and longitudinal monitoring of axial elongation.

Several optical techniques—such as structured light projection and pattern deformation analysis—have been explored to infer axial length indirectly. However, these methods have often lacked sufficient sensitivity, robustness, and calibration accuracy under real-world conditions.

Accordingly, there remains an unmet need for a practical, handheld device capable of accurately estimating axial length and refractive error using compact optical components, minimal operator skill, and real-time image analysis. Such an innovation would dramatically improve access to vision screening, especially in resource-limited and pediatric care settings.

### SUMMARY OF THE INVENTION

The present invention provides a structured light-based optical method and device for measuring ocular axial length and refractive error with high precision and low cost, enabling early detection of myopia and other refractive abnormalities.

The invention combines structured light projection, Modulation Transfer Function (MTF) analysis, liquid lens adjustment, and optional pattern deformation metrics to achieve non-invasive, quantitative assessment of ocular geometry and refractive properties.

### Overview

The system projects a polarized structured light pattern, such as a grid, onto the retina. The reflected pattern is captured by a CMOS imaging sensor through a cross-polarization filter that suppresses reflections from the cornea and crystalline lens, isolating the retinal reflection. A liquid lens is dynamically adjusted over a controlled diopter range to sequentially alter focus. For each lens position, the captured image is analyzed to compute the Modulation Transfer Function (MTF) at one or more spatial frequencies. The focal setting that yields the maximum MTF value corresponds to the eye's optimal focus, which is then mapped to refractive error (D) and axial length (L) via a calibration model based on the Schematic Eye equation.

### Key Innovations

1. Integrated Optical Approach: Combines structured light projection and liquid lens focusing with MTF-based sharpness analysis, enabling precise measurement without the need for interferometry or mechanical contact.
2. Polarization-Based Reflection Isolation: Utilizes orthogonal polarizers to minimize specular reflections and isolate retinal signals, improving measurement accuracy.
3. MTF-Driven Refractive and Axial Estimation: Employs frequency-domain image analysis to determine optimal focus and derive axial length from refractive error using calibrated optical models.
4. Optional Deformation Metrics: Enhances robustness by analyzing grid deformation (spacing, curvature, or distortion) to validate and refine MTF-derived results.
5. Compact and Portable Design: The optical and computational modules are miniaturized for integration into a handheld, battery-powered device suitable for field use and telemedicine.
6. AI-Assisted Calibration and Analysis (optional): Artificial intelligence or machine learning algorithms can be incorporated to automatically recognize grid patterns, correct alignment errors, and optimize MTF computation.

### Advantages Over Prior Art

Eliminates the need for interferometric or ultrasound systems.
Enables accurate measurements using low-cost optical components.
Operates non-invasively and without corneal contact.
Provides rapid, objective, and repeatable assessments.
Suitable for children and untrained operators in clinical or home settings.

The invention therefore enables real-time, quantitative, and accessible measurement of ocular axial length and refractive error, paving the way for scalable myopia screening and monitoring programs globally.

### BRIEF DESCRIPTION OF THE DRAWINGS

FIG. 1 is a schematic diagram illustrating the optical configuration of a structured-light-based ocular measurement system. The system includes a structured light source (104) emitting patterned illumination through a polarizer (106) toward the eye (110). The incident structured light is reflected from ocular surfaces (116), such as the cornea and lens, generating a reflected pattern (118) that travels to an analyzer (120). The analyzer directs the captured signal to an imaging sensor (122), and a processor (128) computes the modulation transfer function (MTF) to derive focus position, refractive error, and axial length.

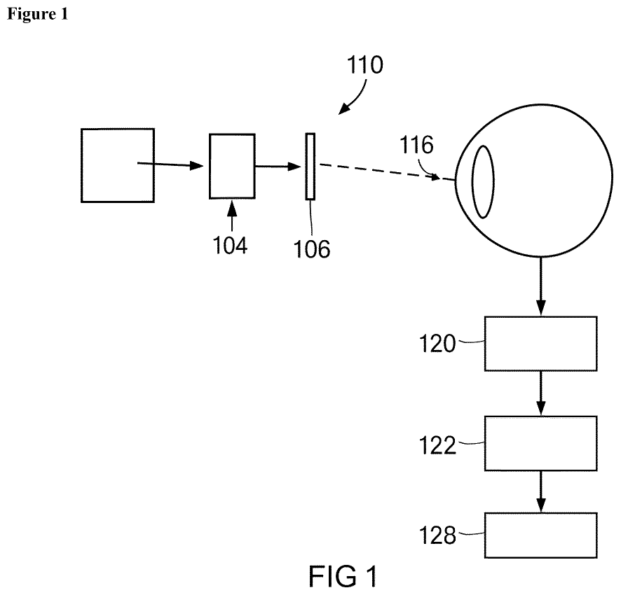

FIG. 2 is a flowchart illustrating the operational workflow of the structured-light-based method for measuring ocular axial length and refractive error. The process begins with system initialization (202), projection of a structured pattern onto the retina (204), capture of the reflected image through an analyzer (206), computation of the modulation transfer function (208), iterative focus adjustment to identify the maximum MTF (210), and display or transmission of the derived axial length and refractive error (212).

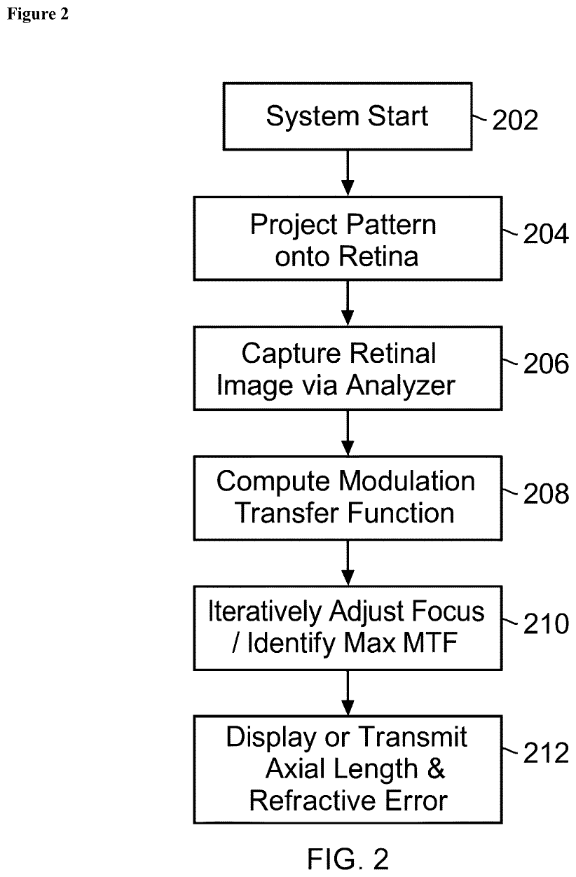

FIG. 3 is a graph illustrating representative Modulation Transfer Function (MTF) curves for emmetropic, myopic, and hyperopic eyes. The vertical axis represents MTF magnitude, and the horizontal axis represents spatial frequency in cycles per degree. The emmetropic eye demonstrates higher MTF values across frequencies, while the myopic and hyperopic curves exhibit reduced or shifted performance, illustrating the focus-dependent variation in image sharpness used to estimate refractive error and axial length.

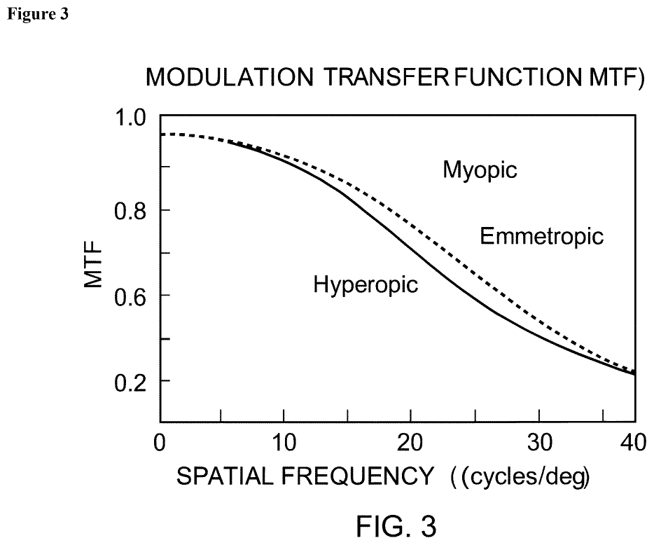

FIG. 4 is a graph illustrating the calibration relationship between liquid-lens focal position, refractive error, and axial length. The solid line represents a linear correlation between lens focal position and refractive error, while the dashed line shows an inverse relationship with axial length. These calibration functions enable estimation of ocular axial length from measured refractive-error values obtained via MTF analysis.

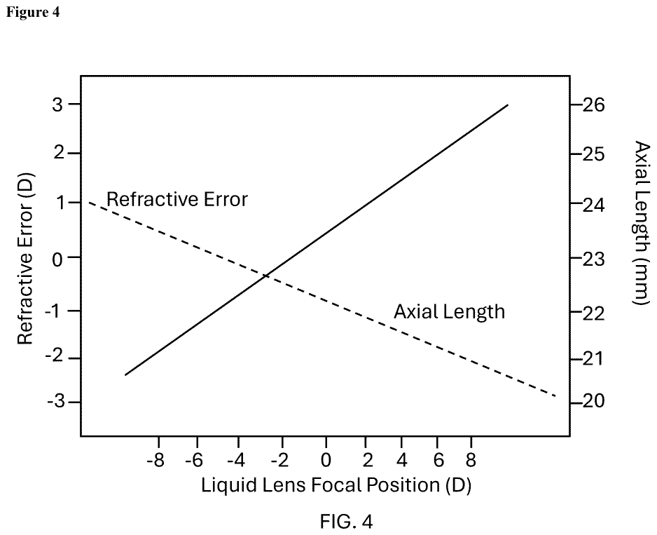

FIG. 5 is a side cross-sectional schematic of a handheld embodiment of the device (502) projecting a structured-light pattern toward a subject eye (520) along an optical path (512). Within the housing, the system includes a structured-light emitter (504), a polarizer (506), a tunable liquid lens (508), and an imaging sensor (510). A processor module (514) computes the MTF-based focus and derives refractive error and axial length, which are shown on a display (516). A connector or control (518) may provide power and/or data.

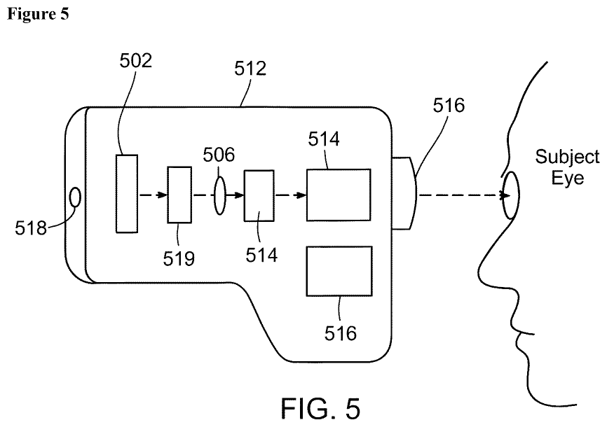

FIG. 6 is a schematic diagram illustrating the deformation-analysis process used to validate and refine MTF-derived results. The system compares the geometry of a reflected structured-light pattern with the reference pattern to determine deviations corresponding to optical distortions. A deformation-mapping module quantifies differences in projected-grid spacing, curvature, or angular displacement, enhancing the accuracy of axial-length and refractive-error estimation.

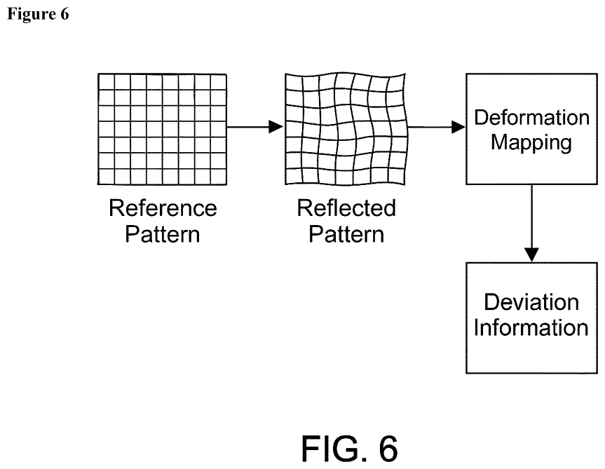

### DETAILED DESCRIPTION OF THE INVENTION

### Overview

The invention provides a non-contact optical method and portable device for measuring ocular axial length and refractive error using structured light projection, Modulation Transfer Function (MTF) analysis, and liquid-lens-based focusing.

The system projects a polarized grid pattern onto the retina, captures the reflected image through an orthogonal polarization filter, analyzes image sharpness in the frequency domain, and determines the eye's optimal focus point. From this focus position, the refractive error (D) and axial length (L) are calculated through calibrated optical relationships derived from the Schematic Eye Model.

### Optical Subsystem

### Structured Light Projection

A structured-light source produces a grid or line-pattern illumination, typically generated by a Digital Micromirror Device (DMD) or Diffractive Optical Element (DOE) illuminated by an infrared or visible LED (e.g., 780-850 nm).

The pattern may be a 5×5 grid with approximately 1 mm spacing at the retinal plane. The emitted light is horizontally polarized to enable cross-polarization detection and to reduce specular reflections.

### Polarization Filtering

To isolate the retinal reflection, a vertical polarizer (analyzer) is positioned in front of the imaging sensor. The orthogonal orientation between illumination and detection polarizers suppresses reflections from the cornea and crystalline lens. Only depolarized or scattered retinal light reaches the sensor, thereby enhancing signal-to-noise ratio and measurement precision.

### Variable Focus Subsystem (Liquid Lens Assembly)

A liquid lens provides electronically controlled focal adjustment over a range of approximately −3 D to +3 D with 0.1 D resolution. The lens curvature is modulated by electrical current or voltage, changing the optical path length of the system without mechanical motion.

During measurement, the lens sequentially steps through preset diopter values. For each setting, the system projects the structured pattern, captures the reflected image, and performs MTF analysis. The focus position yielding maximum MTF represents the optical conjugate of the retinal plane.

### Imaging and MTF Computation

The reflected grid is captured by a CMOS imaging sensor (e.g., 1-2 MP). Each captured frame undergoes two-dimensional Fourier transformation to obtain the frequency-domain representation of the pattern.

The MTF is computed as the normalized modulation of spatial frequency components, typically evaluated at low (≈0.5 cycles/mm) and high (≈2-5 cycles/mm) frequencies.

For each liquid-lens setting, the system calculates:

$$M T F ( f ) = \frac{{I}_{max} ( f ) - {I}_{min} ( f )}{{I}_{max} ( f ) + {I}_{min} ( f )}$$

The optimal focus (Fₒpt) corresponds to the maximum MTF value at high spatial frequencies. The derived refractive error (D) is mapped from Fₒpt using a pre-established calibration curve.

### Calibration and Mapping to Axial Length

A calibration dataset of eyes (or model eyes) with known axial lengths and refractive errors is used to determine constants k and m in the empirical relationship:

$$D = k \cdot {F}_{opt} + m \cdot {M}_{deformation}$$

where
Fₒpt=liquid-lens focal position at peak MTF,
M_deformation=optional grid-deformation metric.

Axial length (L) is then derived from the Schematic Eye Model:

$$L = f ( {P}_{c} , {P}_{l} , D )$$

where P_c and P_l are the corneal and crystalline-lens powers, respectively (typically 43 D and 17 D).

### Optional Deformation Metrics

To enhance robustness, the device may analyze grid deformation as a secondary validation:
1. Measure spacing between adjacent horizontal and vertical grid lines.
2. Compute deviation Δs from the reference pattern.
3. Correlate positive Δs with myopic elongation and negative Δs with hyperopic shortening. These deformation metrics refine or verify the MTF-based results, especially under sub-optimal imaging conditions.

### Processing and User Interface

An embedded microprocessor executes all control and analysis steps: pattern projection, lens control, image capture, FFT/MTF computation, calibration mapping, and result display.

Results shown on a small screen include:
Refractive error (D),
Estimated axial length (L), and
A color-coded risk indicator (e.g., green=normal, yellow=borderline, red=myopia).

The device may also store or transmit data via Bluetooth or Wi-Fi for remote analysis or telemedicine integration.

### AI-Enhanced Operation (Optional Embodiment)

Machine-learning algorithms can be incorporated for:
Automatic grid detection and alignment correction,
Adaptive exposure control and noise suppression,
Population-specific calibration (age, ethnicity, pigmentation).
AI modules may reside locally on the device or in cloud-connected platforms.

### Handheld Device Design

A representative embodiment is a compact, battery-powered handheld unit incorporating:
Structured-light emitter,
Liquid-lens module,
CMOS sensor,
Embedded processor,
Display interface, and
Charging/data port.
The ergonomics allow easy alignment with the patient's eye while maintaining safety standards for optical exposure.

### Use Method

1. Align the device with the subject's eye.
2. Initiate measurement; the system projects the structured pattern.
3. The liquid lens cycles through focus positions automatically.
4. At each position, the MTF is computed and stored.
5. The system identifies Fₒpt, calculates D and L, and displays results.
6. Data may be saved or transmitted for clinician review.

### Advantages and Applications

Non-invasive, contact-free operation.
Accurate detection of myopia, hyperopia, and axial elongation.
Compact, low-cost architecture suitable for school and community screening.
Compatible with pediatric and telemedicine workflows.

### Corneal-Thickness Measurement

In certain embodiments, the system analyzes reflections corresponding to the first and second Purkinje images (P1 and P2) generated by the anterior and posterior corneal surfaces, respectively. The spatial or optical path-length separation between P1 and P2 may be determined through geometric reconstruction, focus-position analysis, or time-resolved imaging to estimate the optical thickness of the cornea. By applying known refractive indices of corneal tissue, the physical corneal thickness can be derived. This measurement may serve as an adjunct diagnostic parameter for corneal ectasia, keratoconus, or surgical screening applications.

1. A method for measuring ocular axial length and refractive error, comprising:
(a) projecting a structured and polarized light pattern onto the retina of an eye;
(b) capturing a reflected image of the pattern using an imaging sensor positioned behind an analyzer that is cross-polarized relative to the illumination;
(c) iteratively adjusting a liquid lens through a series of focal powers;
(d) computing, for each lens position, a modulation transfer function (MTF) from the captured image to quantify image sharpness at one or more spatial frequencies;
(e) identifying an optimal focal position corresponding to a maximum MTF value; and
(f) determining a refractive error and estimating an axial length of the eye from the optimal focal position using a calibrated optical model.

2. A device for measuring ocular axial length and refractive error, comprising:
(a) a structured-light source configured to project a polarized grid or pattern toward a retina;
(b) a liquid lens configured to adjust optical focus over a plurality of diopter settings;
(c) a polarization filter aligned to isolate retinal reflections and suppress corneal or lenticular reflections;
(d) an imaging sensor for capturing reflected pattern images;
(e) a processor configured to compute a modulation transfer function (MTF) for each image, identify a lens position that maximizes the MTF, and derive refractive error and axial length; and
(f) a user interface configured to display results or transmit them to an external device.

3. The method of claim 1, wherein the structured-light pattern comprises a two-dimensional grid of intersecting lines generated by a digital micromirror device (DMD) or diffractive optical element (DOE).

4. The method of claim 1, wherein the liquid lens provides an adjustable focal range between −3 diopters and +3 diopters in increments of approximately 0.1 diopter.

5. The method of claim 1, further comprising calculating one or more deformation metrics from the captured image to validate or refine the measured refractive error or axial length.

6. The method of claim 1, wherein the calibrated optical model includes parameters representing corneal and crystalline-lens powers of approximately 43 and 17 diopters, respectively.

7. The device of claim 2, wherein the polarization filter is oriented orthogonally to the illumination polarization to isolate depolarized retinal light.

8. The device of claim 2, wherein the processor performs a Fourier transform on each captured image to obtain frequency-domain data for computing the MTF.

9. The device of claim 2, further comprising a calibration module storing coefficients that map optimal focus position and deformation metrics to refractive power in diopters.

10. The device of claim 2, further comprising a wireless communication module for transmitting measurement data to a telemedicine or cloud-based analysis platform.

11. The device of claim 2, wherein an artificial-intelligence algorithm is configured to detect grid patterns, correct misalignment, and optimize MTF computation automatically.

12. The device of claim 2, embodied as a portable, battery-powered handheld unit including an optical window, illumination aperture, imaging sensor, and integrated display.

13. A system for early detection of myopia, comprising the device of claim 2 integrated with a telemedicine network or database for longitudinal monitoring of axial-length and refractive-error trends.

14. The device of claim 2, wherein the system further comprises a deformation-mapping module configured to validate MTF-derived results.

15. The method of claim 1, wherein the system determines corneal thickness based on the optical or geometric separation between first and second Purkinje reflections (P1 and P2) from the anterior and posterior corneal surfaces.
